(19) United States
(12) Patent Application Publication
(10) Pub. No.: US 2018/0217159 A1
(43) Pub. Date: Aug. 2, 2018

(54) BIOMARKERS FOR PREDICTING A RESPONSE TO AN IMMUNOMODULATING TREATMENT IN PATIENTS WITH INFLAMMATORY DISEASE

(71) Applicants: UNIVERSIDAD DE CHILE, Santiago (CL); PONTIFICIA UNIVERSIDAD CATÓLICA DE CHILE, Santiago (CL)

(72) Inventors: Rodrigo Antonio NAVES PICHUANTE, Santiago (CL); Claudia CÁRCAMO RODRÍGUEZ, Santiago (CL)

(21) Appl. No.: 15/572,279

(22) PCT Filed: May 6, 2016
(86) PCT No.: PCT/CL2016/050022

§ 371 Date: Apr. 9, 2018

(30) Foreign Application Priority Data
May 7, 2015 (CL) 1213-2015

(51) Int. Cl. G01N 33/68

(52) U.S. Cl. CPC G01N 33/6866; G01N 33/6869; G01N 2800/52; G01N 2333/5428; G01N 2333/57; G01N 2333/54; G01N 2800/24

(57) ABSTRACT

The present invention provides a method and a kit for predicting the response to an immunomodulatory treatment in patients that present an inflammatory disease such as multiple sclerosis, which are based on the quantification of the plasma levels ratio of cytokines IL-17F, IFN-γ and IL-10 in a biological sample from the patient taken before initiating the treatment.

### TECHNICAL FIELD

The present invention relates to the technical field of immunology, neurology and human health and particularly, provides a method and a kit for predicting response in patients with inflammatory disease, specifically with multiple sclerosis, to the treatment with an immunomodulatory agent.

### BACKGROUNDS OF THE INVENTION

The multiple sclerosis (MS) is a chronic inflammatory autoimmune disease in which the individual develops an immune response against his own components of the central nervous system. This pathology affects young adults between 20 and 40 years old and constitutes the main non-traumatic cause of physical disability and incapacity in this segment of the population (Hohlfeld, R. European journal of immunology 39.8 (2009):2036-2039), generating a significant social and economic impact for patients and the health system.

The etiology of the disease is multifactorial, occurring in people with a genetic susceptibility associated to environmental factors (Korn, T. Journal of neurology 255.6 (2008):2-6; International Multiple Sclerosis Genetics Consortium, and Welcome Trust Case Control Consortium 2. Nature 476.7359 (2011):214-219). The clinical manifestations of Multiple Sclerosis include visual and sensory alterations, walking deterioration, paresis (muscular weakness), spasticity (increase of muscle tone), ataxia (coordination loss) and fatigue. Over time, these symptoms accumulate and appear other related disorders such as urinary incontinence, cognitive alterations and depression (Ruet A. and cols. Neurology 80.16 (2013):1501-1508; Ziemssen, T. Journal of the neurological sciences 277 (2009): S37-S41). Due to early onset of the symptoms and its chronic nature, the MS severely affects life quality of patients and determines a significant decrease in labor productivity and considerable economical costs for the patient and the health system in a long period of time (Asche, C. and cols. Journal of managed care pharmacy 16 (2010):703-712; Naci, H and cols. Pharmacoeconomics 28.5 (2010): 363-379).

An important characteristic of the MS is its high clinical heterogeneity. Initially the disease occurs with a first episode of neurological disturbance that it is known as clinical isolated syndrome (CIS). Then, 85% of patients develop MS in its Relapsing-Remitting (RRMS) clinical form, which consists in neurological alteration attacks (outbreaks) followed by total or partial recovery periods between an episode and another (Hurwitz, B. Annals of Indian Academy of Neurology 12.4 (2009):226). In the course of 10 to 15 years of evolution, about 40-50% of the patients with RRMS will develop Secondary Progressive MS (SPMS), accumulating disability without remission. A lower percentage (15%) of patients presents since the beginning of the disease a form of Primary Progressive MS (PPMS) characterized by the progressive accumulation of neurological damage without relapsings (Mahad, D. and cols. The Lancet Neurology 14.2 (2015):183-193). This is how we have three main forms of evolution: multiple sclerosis RRMS, SPMS and PPMS.

The MS does not have a cure but the use of modifying therapies of the disease has a significant effect on the progression of the same, although only in the clinical form RRMS. In the market exists several options of this type of immunomodulatory treatments such as: glatiramer acetate, type I interferons (IFNs), fingolimod and natalizumab. However, the effectiveness and the adverse effects associated to each one of them are different and the response to these therapies varies depending on the aggressiveness that the disease develops.

The systemic administration of IFN-β, a type I interferon, constitutes one of the first-line therapies widely used around the world due to its proved effectiveness, safety and tolerability. (Rudick, R. and cols. Experimental cell research 317.9 (2011):1301-1311; George, P. and cols. Pharmacology & therapeutics 135.1 (2012): 44-53). There are several commercial preparations available in the market of IFN-β1a: Avonex® from Biogen, US: Rebif® from Merck-Serono/Pfizer, Switzerland; and IFN-β1b: Betaseron®/Betaferón® from Bayer HealthCare Pharmaceuticals, Germany; and Extavia® from Novartis, Switzerland. It has been established that the treatment with IFN-β decreases the outbreaks rate in approximately 30%, extending the remission periods of the disease, decreasing the inflammatory injuries formation in the central nervous system of patients and slowing down the pathological progression (Schwid, S. and cols. Clinical therapeutics 29.9 (2007):2031-2048). Despite this, the treatment with IFN-β is just partially effective and 40% of the patients does not respond, respond weakly or discontinue the treatment due to collateral effects or exacerbations of the disease (Shimizu, Y. and cols. Journal of neurology 255.2 (2008): 305-307; Warabi, Y. and cols. Journal of the neurological sciences 252.1 (2007): 57-61). Even more, a current problem originated as a result of this is that it does not exist a way to know in advance if the treatment with IFN-β will be effective in a determined patient. This situation is extremely important due to it is required a period of 1 to 2 years of a clinical follow-up to verify the effectiveness of the treatment, which favors the neurological damage accumulation in those patients that respond negatively to the treatment (Rio, J. and cols. Nature Reviews Neurology 5.10 (2009): 553-560). Consequently, due to the chronic, progressive and destructive nature of RRMS, to the risk of the adverse effects and to the considerable costs of the therapy for the patient and the health system, there is a great need for identifying biomarkers that can predict the response to the treatment for this disease before initiating therapy. Despite of the efforts of several groups of researchers, until now it has not been possible to validate a reliable and reproducible biomarker for these purposes (Rudick, R. and cols. Neurology 79.6 (2012): 498-499).

Scientific literature related to immunological basis of multiple sclerosis has described that sub-populations of T helper cells (Th) such as Th1, Th2 and Th17, as well as the selective profile of cytokines that these cells produce, are crucial components of the inflammatory process associated to the disease development. IFN-γ is a cytokine produced by Th1 lymphocytes, that in cooperation with cytokines IL-17A and IL-17F, produced by Th17 lymphocytes, are proinflammatories and they have been associated with the beginning and progression of the disease (Steinman, L. The Journal of experimental medicine 205.7 (2008):1517-1522; Pierson, E and cols. Immunological reviews 248.1 (2012):205-215). On the other hand, IL-10 produced by Th2 lymphocytes has an anti-inflammatory activity and it has been correlated with an improvement of symptoms. (Imitola, J. and cols. Pharmacology & therapeutics 106.2 (2005): 163-177).

The family of interleukins 17 (IL-17) comprise a set of six different cytokines including from IL-17A to IL-17F. Even though IL-17A (commonly denominated IL-17) and IL-17F possess a high identity of amino acid sequence, each one of them performs different biological functions in the development of certain pathologies, including MS. Whereas IL-17A is involved in autoimmunity, inflammation, cancer and defense against bacterial and fungal infections; the IL-17F participates in defense mechanisms of the host in mucosa, according to the published by Yang X. O. and cols. The Journal of experimental medicine 205.5 (2008): 1063-1075 and by Iwakura Y. and cols. Immunity 34.2 (2011): 149-162.

Several studies have analyzed changes in cytokine levels in the serum or plasma of patients with RRMS throughout the treatment with IFN-β, as a manner of defining biomarkers of therapeutic effectiveness (Graber, J. and cols. Journal of neuroimmunology 185.1 (2007):168-174; Wiesemann, E. and cols. Clinical Immunology 128.3 (2008):306-313). Nevertheless, few studies have addressed the task of identifying predictive biomarkers of therapeutic response present in the plasma of patients before beginning the treatment. It has been established that part of the beneficial effects of the treatment with IFN-β in RRMS patients are correlated with a decrease of IL-17A and an increase in the secretion of IL-10 in the serum of RRMS patients (Kvarnström, M. and cols. Journal of the neurological sciences 325.1 (2013): 79-85). However, there are conflicting results regarding the predictive value of IL-17A in response to IFN-β. In fact, it has not been detected differences in basal levels (before initiating treatment with IFN-β) of IL-17A among patients that respond positively or negatively to the treatment. Journal of neuroimmunology 254.1 (2013): 131-140).

As previously mentioned, IL-17F is one of the characteristic cytokines secreted by Th17 lymphocytes. The scientific articles published by Axtell, R. C. and cols. Nature medicine 16.4 (2010): 406-412; and Lee L. F. and cols. Science translational medicine 3.93 (2011): 93ra68, have reported a positive correlation between high levels of IL-17F and lack of response to the treatment with IFN-β in patients with RRMS. These results were used as basis to the patent document U.S. Pat. No. 8,828,668, however, in subsequent scientific documents carried out in larger groups of patients such as the publication of Bushnell S. E., and cols. Neurology 79.6 (2012): 531-537 and the publication of Hartung H. P. and cols. JAMA neurology 70.8 (2013): 1017-1021, it has not been possible to validate those results and different conclusions have been reached suggesting that IL-17F is not a valid predictive biomarker of the effectiveness of the treatment with IFN-β1a as IFN-β1b.

Regarding other cytokines analyzed in the search of predictive markers of response to treatments in patients with multiple sclerosis, a study published by Bartosik-Psujek H. and Zbigniew S. Clinical neurology and neurosurgery 108.7 (2006): 644-647, reported that the presence of low levels of IL-10 before the beginning of the therapy predicts a positive response to the treatment with IFN-β. However, the subsequent publication of Wiesemann E. and cols. Clinical Immunology 128.3 (2008): 306-313 described that no differences were found in IL-10 levels before the treatment in patients that respond positively or negatively to the treatment; and besides it suggests that IL-10 could have a predictive value only in combination with the analysis of other lymphoid surface markers such as CD86, CD40 and PD-L2. According with the aforementioned, the work of Dhib-Jalbut S. and cols. Journal of neuroimmunology 254.1 (2013): 131-140, did not reveal significant differences in IL-10 levels produced by peripheral blood mononuclear cells (PBMC) obtained from patients that respond positive or negatively to the treatment with IFN-β1b and that are subsequently stimulated.

On the other hand, studies related to IFN-γ such as the Petereit H. F. and cols. Multiple sclerosis 8.6 (2002): 492-494, have shown that the low basal concentration of this cytokine predicts a favorable response to the therapy with IFN-β, however the subsequent study of Graber J. J. and cols. Journal of neuroimmunology 185.1 (2007): 168-174 does not report significant differences in this cytokine in patients that respond positive and negatively to the treatment. In the work of Bustamante M. F. and cols. Clinical & Experimental Immunology 171.3 (2013): 243-246 it was determined the level of IFN-γ, IL-17A, IL-17F, IL-10 and IL-4 in activated PBMC cell supernatants obtained from patients that respond positively or negatively to the treatment with IFN-β, however no differences were found in both groups. Therefore, existing investigations in the state of the art focused in the search of cytokines that can predict individually the response of the patients to the treatments have not been successful until now.

Among patent documents related to predictive markers of the clinical response to the treatment with modulating agents in patients with multiple sclerosis we can find the patent application MX2013003929 which describes a method to predict the response to the therapy with glatiramer acetate based in the concentration measurement of cytokines IL-17A, TNF-α, IL-2 and IFN-γ individually in activated PBMC cells supernatants of the patients and its comparison with control values. On the other hand, patent U.S. Pat. No. 8,759,302 also describes a method to assess the response to the same treatment, based on the assessment of the individual concentration of cytokines IL-10, IL-17A, IL-18, TNF-α, among others, and the relation among them.

Even though glatiramer acetate and IFN-β treatments are the main two groups of drugs used in the treatment of multiple sclerosis, their action mechanisms are completely different. According to the reported by Comi G. and Moiola L. Neurologia-Barcelona 17.5 (2002):244-258 and Yong V. W Neurology 59.6 (2002):802-808, IFN-β exerts a main activity in the blood-brain barrier and induces an inhibition of the expression of adhesion molecules and chemokines receptors which leads to a decrease in the traffic of inflammatory cells to the central nervous system. In addition, this treatment inhibits the differentiation of Th17 lymphocytes and proinflammatory cytokines production such as IL-17 and osteopontin (Chen, M. and cols. European journal of immunology 39.9 (2009): 2525-2536 and Ramgolam, V. and cols. The Journal of Immunology 183.8 (2009): 5418-5427). In contrast, the glatiramer acetate produces an imperceptible effect in the blood-brain barrier and on the contrary, induces to the activation of specific T helper cells of phenotype Th2 that would migrate to central nervous system, performing anti-inflammatory and suppressing activity (Neuhaus, O. and cols. Neurology 56.6 (2001): 702-708 and Sela, M. and cols. Expert opinion on pharmacotherapy 2.7 (2001): 1149-1165).

In the light of the exposed backgrounds, there exists an important need—non-solved until today—for determining biomarkers capable of predicting the therapeutic response to the treatment with immunomodulating agents in patients with multiple sclerosis before such treatment is initiated.

### CONTENTS OF THE INVENTION

An object of this invention refers to an ex vivo method to predict the response to an immunomodulatory treatment in patients with inflammatory disease that comprises the following stages:
to obtain a biological sample from a patient;
to quantify in said biological sample from the patient at least one predictive biomarker selected from the group that consists in the ratio between the concentrations of cytokines IFN-γ/IL-17F, IL-17F/IL-10, IL-17F/IFN-γ and IL-10/IL-17F;
to compare the value of the ratios between said cytokines of the biological sample from the patient with representative values of these ratios measured in samples of a patient's population that respond negatively to the treatment;
to predict a positive response to the treatment based on a greater value of the ratio IFN-γ/IL-17F or IL-10/IL-17F or a lower value of the ratio IL-17F/IL-10 or IL-17F/IFN-γ in the sample of the patient, in comparison to the representative value in the sample of the patients population that respond negatively to the treatment.

In a preferred modality of the invention, the inflammatory disease is an autoimmune disease, and in particular is multiple sclerosis.

To predict the response to the immunomodulatory treatment, the following stages are carried out additionally:
to calculate the ratio of the concentration of cytokines IFN-γ/IL-17F and IL-17F/IL-10 by mathematical division;
to compare ratios value with a determined cut-off value by a predictive modeling method in patient's populations that respond positive and negatively to the immunomodulating treatment; and
to predict the positive response to the immunomodulatory treatment when the value of the ratio IFN-γ/IL-17F measured in the patient is greater than or equal to the determined cut-off value or when the value of the ratio IL-17F/IL-10 measured in the patient is lower than the determined cut-off value.

Another object of this invention refers to a kit for predicting the response to an immunomodulating treatment in patients with inflammatory disease, that comprises the following:
means to obtain a biological sample from the patient;
means to quantify in said biological sample from the patient at least one predictive biomarker selected from the group that consists in the ratio between the concentrations of cytokines IFN-γ/IL-17F, IL-17F/IL-10, IL-17F/IFN-γ and IL-10/IL-17F;
means to compare the value of the ratios between said cytokines concentrations of the biological sample from the patient with representative values of these ratios measured in samples of a patient's population that respond negatively to the treatment;
means to predict a positive response to the treatment based on a greater value of the ratio IFN-γ/IL-17F or IL-10/IL-17F or a lower value of the ratio IL-17F/IL-10 or IL-17F/IFN-γ in the sample of the patient, in comparison to the representative value in the sample of the patients population that respond negatively to the treatment.
a brochure with instructions to use said means.
   Said kit is preferably used to assess patients with multiple sclerosis.

The immunomodulatory treatment to which the method and the kit of this invention refers includes a drug selected from the group that consists in a type I interferon, glatiramer acetate, natalizumab, fingolimod or a combination of these. The biological sample from the patient can be selected from the group that consists in blood, blood plasma, blood serum, peripheral blood mononuclear cells, cerebrospinal fluid and urine.

### BRIEF DESCRIPTION OF THE FIGURES

FIG. 1 shows the level of IFN-γ, IL-10, TGF-β, IL-17A, IL-17F and the ratio of IFN-γ/IL-17F in samples of blood plasma obtained from patients with clinical isolated syndrome (CIS, n=21), relapsing-remitting multiple sclerosis (RRMS, n=34), secondary progressive multiple sclerosis (SPMS, n=11), primary progressive multiple sclerosis (PPMS, n=17) and healthy controls patients (HC, n=17) by the means of ELISA tests. The horizontal line represents median of the values.

FIG. 2 represents the association between the ratio of the levels of cytokines IFN-γ/IL-17F measured in blood plasma in patients with relapsing-remitting multiple sclerosis (RRMS) with the disease activity determined by imaging analysis by nuclear magnetic resonance and the relapse rate.

FIG. 3 shows the quantification of level of cytokines IL-17F, IFN-γ, IL-10, and the ratios between the concentration of cytokines IFN-γ/IL-17F and IL-17F/IL-10 in RRMS patients that after a year of treatment with IFN-β presented a positive response (Resp, responders) or negative response (NR, non-responders) to the treatment. In addition, it shows the correlation between the plasma levels of IFN-γ and IL-17F in patients responding positively to the treatment.

FIG. 4 shows ROC curves analysis for the ratios between IFN-γ/IL-17F and IL-17F/IL-10 in RRMS patients that respond positively and negatively to the treatment (AUC, Area Under the Curve) (*: cut-off value).

FIG. 5 shows the ratio between the levels of cytokines IL-17F/IFN-γ in RRMS patients that after a year of treatment with IFN-β presented a positive response (Resp, responders) or negative response (NR, non-responders) to the treatment; and the ROC curve analysis for the ratio between concentrations of cytokines IL-17F/IFN-γ (AUC, Area Under the Curve) (*: cut-off value).

### DETAILED DESCRIPTION OF THE INVENTION

The present invention refers to an ex vivo method for predicting the response to an immunomodulatory treatment in patients that develops an autoimmune inflammatory disease such as multiple sclerosis. Particularly, the present method is based on the quantification of biomarkers predicting response to the treatment that are related to cytokines produced by Th1, Th2 and Th17 lymphocytes, more precisely it refers to biomarkers quantification based on a ratio of the cytokine levels of IL-17F, IFN-γ e IL-10 measured in a biological sample from patients with relapsing-remitting multiple sclerosis. Predictive biomarkers of the present invention include: IFN-γ/IL-17F, IL-17F/IL-10, IL-17F/IFN-γ or IL-10/IL-17F. Said ratios are calculated by mathematical division of the concentrations of cytokines present in a biological sample from the patient, preferably venous blood (blood serum or plasma), cerebrospinal fluid or urine.

In the state of the art, the measurement of individual cytokine level before the beginning or during the treatment with immunomodulatory agents is not associated to the response of the patients to the treatment. Nevertheless, and as part of this invention, values of the ratios between the before-mentioned cytokines concentrations are directly associated to the response of the patients to the treatment. In a preferred modality, ratios that are associated to the effective prediction of said response to the treatment are IFN-γ/IL-17F and/or IL-17F/IL-10. The response to the treatment may be positive or negative for the patient, where positive response prediction is made based on the use of a statistical method of predictive modeling.

In addition to that, the present invention refers to a kit for predicting the response to an immunomodulatory treatment in patients with inflammatory disease based on biomarkers determination through the method described in the same, where said kit comprises means to obtain a biological sample from the patient, means to quantify in said sample at least one predictive biomarker selected from the group that consists in the ratios between the concentrations of cytokines IFN-γ/IL-17F, IL-17F/IL-10, IL-17F/IFN-γ and IL-10/IL-17F; means to compare the values of said ratios measured in the patient with representative values of the same ratios measured in samples of a patients population that respond negatively to the treatment and means to predict a positive response to the immunomodulatory treatment based on a greater value of the ratio IFN-γ/IL-17F or IL-10/IL-17F, or a lower value of the ratio IL-17F/IL-10 or IL-17F/IFN-γ in the patient's sample, in comparison to the representative value in the patients population sample that respond negatively to the treatment; besides of a brochure with instructions for its use. For quantifying at least one predictive biomarker said kit comprises means to quantify the concentrations of cytokines IFN-γ, IL-17F and IL-10 in the biological sample from the patient and means to process mathematically and statistically the obtained values and calculate the probability of the patient for responding to the immunomodulatory treatment. The means for taking a biological sample from the patient include—no restrictions—syringes, cannulas, catheters, tubes, hoses and other elements known by any expert on the matter. The means to quantify the cytokines include—no restrictions—reagents known in the state of the art such as specific antibodies capable of recognize said cytokines or other reagents of common use in ELISA techniques, flow cytometry, PCR in any of its variants, microarrays, among other techniques widely known by any expert on the matter. The means to process mathematically and statistically the measured values include—no restriction—calculators, computer applications or algorithms or any other way that allows the automatic calculation of the concentrations ratios of the mentioned cytokines and the patient's probability of responding to the immunomodulating treatment.

For the purposes of the present invention, the following terms are understood as defined as follows.

The term “inflammatory disease” refers mainly to autoimmune pathologies, which are caused by the immune system when activated in response to components of the own body. This type of diseases is characterized by the presence of T and B lymphocytes that abnormally activates in response to organism's self molecules such as proteins, lipids, nucleic acids, among others; causing damage, inflammation and organs damage. Mammals immune system uses complex mechanisms to generate protective responses against pathogens such as virus and bacteria, while at the same time it prevents the responses against self molecules. The effector function of the immune system is mediated by T lymphocytes, among other cells types, which are classified in T helper cells Th1, Th2, Th3, Th17, etc. The effector response of T lymphocytes include the production or secretion of cytokines that characterize each T lymphocyte lineage or subtype. Among the cytokines that produce the different mentioned lineages, Th1 lymphocytes when activated mainly secrete IFN-γ, whereas Th2, Th3 and Th17 lymphocytes produce IL-10, TGF-β and IL-17, respectively.

There are diverse inflammatory diseases where Th17 lymphocytes have a key role in the disease pathogenesis. Since methods of the present invention are associated with biomarkers for predicting the response to immunomodulatory therapies in diseases associated to Th17 lymphocytes, the methods are widely applicable to chronic inflammatory diseases of interest, no limitation, such as: multiple sclerosis, in all subtypes, optic neuritis, amyotrophic lateral sclerosis, Alzheimer's disease, Parkinson's disease, psoriasis, systemic lupus erythematosus, ulcerative colitis, Crohn's disease, ankylosing spondylitis, rheumatoid arthritis, diabetes mellitus type 1, asthma, chronic obstructive pulmonary disease, chronic hepatitis, atherosclerosis, metabolic syndrome and obesity.

The term biomarker (s) or marker (s) refers to, no limitation, cytokines, chemokines, growth factors, proteins, peptides, nucleic acids, oligonucleotides and metabolites along with its respective variants, either mutations, polymorphisms, artificial modifications, fragments, sub-units or degradation products or any other related analyte. Markers also can refer to non-blood factors or non-analytic physiological factors of health condition and/or other non-determined factors in a biological sample such as clinical parameters.

The term immunomodulatory or immunosuppressive treatment comprises therapies that use interferon β (IFN-β) (in trade names such as Avonex, Betaseron, Rebif), glatiramer acetate (Copaxone), fingolimod (Gilenya) and anti-integrin antibodies such as natalizumab (Tysabri). Particularly, for multiple sclerosis, other immunosuppressive agents include methylpredinisolone, steroids, methotrexate, cladribine and cyclophosphamide.

The term ratio refers to the quotient obtained dividing the first number of the comparation by the second. In the present invention, said numbers correspond to the concentration of cytokines IL-17F, IFN-γ and IL-10. For instance, the IFN-γ/IL-17F ratio refers to the quotient between the concentration of cytokines IFN-γ and IL-17F.

The term patient, subject or individual is either a human mammal or a mammal that represents an animal model.

The term analysis corresponds to the comparisons of a set of measurements made in a patient's sample and a sample or a set of control or standard samples. Comparisons determine if the values of these measurements are equal or different between both sample types. Determinations and comparisons are performed by using techniques and statistical methods known in the state of the art.

In reference to the method for predicting the response to an immunomodulatory treatment in patients with an inflammatory disease, the biological sample may include—no limitations—whole blood or its derivatives such as serum, plasma, platelets, erythrocytes and leukocytes. Likewise, the sample may include single cells or a plurality or fragments thereof, apart from tissue biopsies, synovial fluid, cerebrospinal fluid, lymphatic fluid, ascitic fluid, interstitial fluid, extracellular fluid, saliva, mucus, sputum, semen, urine, perspiration, gingival crevicular fluid or any other body fluid. Preferably, the sample comes from serum or blood plasma, cerebrospinal fluid, peripheral blood mononuclear cells (PBMC) or supernatants from PBMC culture. The sample can be obtained from a patient by venous puncture, excretion, ejaculation, massage, biopsy, vacuuming, washing or any other clinical procedure known in the state of the art.

The term standard refers to any control sample(s) or control value(s) that serve(s) as reference. The control sample may come from one or more healthy patient or from patients with multiple sclerosis that has not been treated with IFN-β. The choice of each control type in trials is explained for each case.

In the context of this invention, it is considered that the level of concentration, expression or activity of the marker significantly differs of the standard if it is determined that is improbable that this deviation occurs in a subject or control group. An altered expression level may correspond either to an increased concentration, expression or activity of the marker or a decreased concentration, expression or activity of the marker regarding the standard concentration level, expression or activity.

The present invention refers to different subtypes of multiple sclerosis that are described in the reference Compston A. and cols. The Lancet 359 (2002) 1221-1231 and Hurwitz, B. Annals of Indian Academy of Neurology 12.4 (2009): 226-230.

The term clinical isolated syndrome (CIS) refers to patients that present a first neurological episode caused by demyelination process in central nervous system (Miller, D. and cols. The Lancet Neurology 4.5 (2005): 281-288).

The term relapsing-remitting multiple sclerosis (RRMS) refers to patients that experiment sporadic exacerbations or relapses, as well as remission periods where no symptoms are observed (Hurwitz, B. Annals of Indian Academy of Neurology 12.4 (2009): 226-230).

The term secondary progressive multiple sclerosis (SPMS) refers to RRMS patients that usually progress to this subtype of the disease, where patients experience a gradual increase of neurological disability with or without clinical relapses (Mahad, D. y cols. The Lancet Neurology 14.2 (2015): 183-193).

The term primary progressive multiple sclerosis (PPMS) refers to patients that experience a continuous progression of the neurological deficiencies from its beginning, without relapses or remissions (Mahad, D. y cols. The Lancet Neurology 14.2 (2015): 183-193). In all subtypes of the disease, neurological deficiencies are assessed by the Expanded Disability Status Scale (EDSS) according to the document Kurtzke J. F. Neurological Sciences 21.6 (2000): 339-341.

On the other hand, the term healthy control (HC) refers to healthy patients that do not suffer from multiple sclerosis.

The present invention comprises a method for predicting the response to an immunomodulatory treatment in a patient with chronic autoimmune inflammatory disease, particularly relapsing-remitting multiple sclerosis. The response to the treatment can be positive or negative depending on if this provides a clinical benefit or not for the patient, respectively. The positive response to the treatment is defined based on the following clinical criteria and images taken by magnetic resonance imaging (MRI), assessed within at least a year of treatment: 1) Absence of recurrence or relapse; 2) a progression in EDSS scale no more than one point; 3) presence at the most of a new or enlarging T2-weighted MRI lesion; and 4) no new gadolinium-enhancing T1 MRI lesion injury. In the documents published by Rovira A. and cols. Neurologia 25.4 (2010): 248-265 and by Cordovez, J. and cols. Revista chilena de radiologia 19.4 (2013): 156-164 recommendations are shown for the assessment of images taken by magnetic resonance imaging.

For practical purposes of the present invention the following stages are considered:

### Sample Obtaining:

A biological sample from the patient is collected such as venous blood or cerebrospinal fluid in lab tubes that contain an anticoagulant, preferably the anticoagulant is EDTA. Plasma samples are obtained by centrifugation at 2000×g during 10 minutes at 4° C., it can be divided in aliquots to be stored at −80° C. until the procedure is performed. Other methods for obtaining the plasmatic fraction from patient's sample are within the invention scope. In a preferred modality the sample is venous blood.

### Cytokines Measurement:

Cytokines quantification in plasma obtained from patient's sample can be performed by different methods described in the state of the art. One of the preferred methods for cytokines quantification is an ELISA assay. This assay can be performed by using kits that are commercially available or monoclonal antibodies and reagents used for these purposes which are obtained by anyone with experience in this matter. Among the commercial kits that can be used for cytokines quantification we can find eBioscience and R&D Systems Inc. Usually, measurements are performed in duplicate.

Other methods for cytokines quantification in the patient's sample that are included in the scope of the invention are microarrays assays, Luminex, flow cytometry, CBA (Cytometric Bead Array, BD Biosciences), PCR and its variants, among others, which are known by any expert on the matter.

### Analysis of Predictive Modeling and Probability Calculation of Responding to the Immunomodulatory Treatment by Statistical Methods:

In addition to cytokines quantification and the determination of IFN-γ/IL-17F, IL-17F/IL-10, IL-17F/IFN-γ or IL-10/IL-17F as predictive biomarkers, the present method comprises calculating patient's probability of responding successfully to the treatment, based on the comparison with a cut-off value determined by a statistical method of predictive modeling for one or more biomarker(s).

Different analytical statistical models can be used and compared to obtain a predictive algorithm. These statistical models are known by any person with experience and knowledge in the matter. In general terms these models include, but are not limited to: cluster analysis, factor analysis, discriminant function analysis (DFA) and multidimensional scaling (MDS).

These models can be classified in two types:
1. Models of multivariate statistical analysis, which include, but are not limited to: linear regression analysis, variance and covariance analysis, logistic regression analysis, multivariate analysis of ROC (Receiver Operated Characteristic) curves, multidimensional scaling (MDS), linear discriminant analysis (LDA) and partial least-squares discriminant analysis (PLS-DA).
2. Supervised or machine learning methods, which include, but are not limited to: decision trees such as classification and regression tree (CART), multiple additive regression tree (MART); Prediction and aggregation methods such as Boosting (for example, AdaBoost algorithm) or Bagging; K-nearest neighbors method (KKN) or Weighed k-nearest neighbors method (WKNN); Nearest shrunken centroid method (NSC) and its PAM application (Predictor Analysis of Microarrays); Support vector machine method (SVM); Random forest (RF); Bayesian networks, neural networks and analysis of formal concepts. (Galois).

All these models can be used as part of the analytical process to build a multivariate classification that allows the identification of a set of variables (ratios of cytokines in the present invention) that best differentiate between individual groups (for example, patients that respond positively versus patients that respond negatively, in the present invention). This classification is performed firstly in a cohort of patients that serves as reference for later determining, applying the generated predictor model, if a new patient is classified as responder (that responds positively) or non-responder (that responds negatively) to the treatment. Then, this can be used for predicting the response to the immunomodulatory therapy with IFN-β.

Examples of the invention performance are presented hereunder, which have been included in order to illustrate the invention, its preferred modalities and comparative examples, however, under no circumstance must be considered to restrict the scope of the patent application, which is delimited only by the content of the claims that are attached hereto.

### EXAMPLES

### Example 1: The Levels Ratio Between Cytokines Produced by Th1 and Th17 Lymphocytes is Altered in Blood Plasma of Different Subtypes of Patients with Multiple Sclerosis

First of all, it was determined if cytokines produced by lymphocytes Th1, Th2, Th3 and Th17 from patients with multiple sclerosis and healthy controls (HC) could serve as biomarkers to distinguish between different subtypes of the disease and healthy controls. In order to avoid the influence of therapeutic interventions on real immune system disturbances, only untreated patients were analyzed.

Eighty-three patients were included in the research, of which 21 of them were diagnosed with clinical isolated syndrome (CIS) and 62 with multiple sclerosis, of which 34 of them were classified as RRMS, 11 as SPMS and 17 as PPMS. Seventeen healthy individuals were used as healthy control group (HC). Multiple sclerosis diagnosis and the assessment of the clinical course were defined according to the revised McDonald criteria (Polman C. H and cols. Annals of neurology 58.6 (2005): 840-846) and Lublin-Reingold (Lublin F. D. and Reingold S. C. Neurology 46.4 (1996): 907-911), respectively. Clinical examination and the magnetic resonance imaging analysis of brain and spinal cord were performed in all the patients before inclusion in the study. Disability was assessed by Kurtzke's Expanded Disability Status Scale (EDSS) (1983): 1444-1444). Patients with symptoms of acute systemic inflammation and inflammatory neurological diseases other than multiple sclerosis and patients receiving immunosuppressive or immunomodulating treatment within the previous three months were excluded. Blood samples were collected before beginning of IFN-β treatment. The demographic and clinical characteristics of patients and healthy controls are shown in Table 1.

TABLE 1
Clinical and demographic characteristics of patients
with multiple sclerosis and healthy controls.

| | CIS | RRMS | SPMS | PPMS | HC |
|---|---|---|---|---|---|
| Characteristics | (n = 21) | (n = 34) | (n = 11) | (n = 17) | (n = 17) |
| Male/Female | 7/14 | 10/24 | 3/8 | 7/10 | 7/10 |
| Age, mean in years (SD) | 31.7 | 32.8 | 42.2 | 56.4 | 33.6 |
| | (8.15) | (9.9) | (10.1) | (8.82) | (12.4) |
| Disease duration, | 0.7 | 4.7 | 11.7 | 15.2 | — |
| mean in years (SD) | (0.98) | (4.2) | (5.7) | (10.6) | |
| Value EDSS, mean (SD) | 1 | 1 | 6 | 6 | — |
| | (0.95) | (0.93) | (1.53) | (2.2) | |

Abbreviations: CIS (Clinical Isolated Syndrome), RRMS (Relapsing-Remitting Multiple Sclerosis), SPMS (Secondary Progressive Multiple Sclerosis), PPMS (Primary Progressive Multiple Sclerosis), HC (Healthy Controls), EDSS (Expanded Disability Status Scale), SD (Standard Deviation).

Plasma samples obtained from patients and healthy controls were tested for IFN-γ, IL-10, TGF-6, IL-17A and IL-17F. For these purposes, venous blood sample from patients and healthy controls was collected in Vacutainer tubes (BD Biosciences) containing EDTA. The plasma obtained by centrifugation at 2.000×g during 10 minutes at 4° C., was aliquoted and stored at −80° C. until assessed. Levels or plasmatic concentrations of IFN-γ, IL-10, TGF-β, IL-17A and IL-17F were measured by ELISA assays using eBioscience commercial kits or R&D Systems. All samples were tested in duplicate. Differences between different groups were statistically analyzed using the nonparametric test Mann-Whitney with the GraphPad Prism 5.0 program. It was considered values p<0.05 as statistically significant.

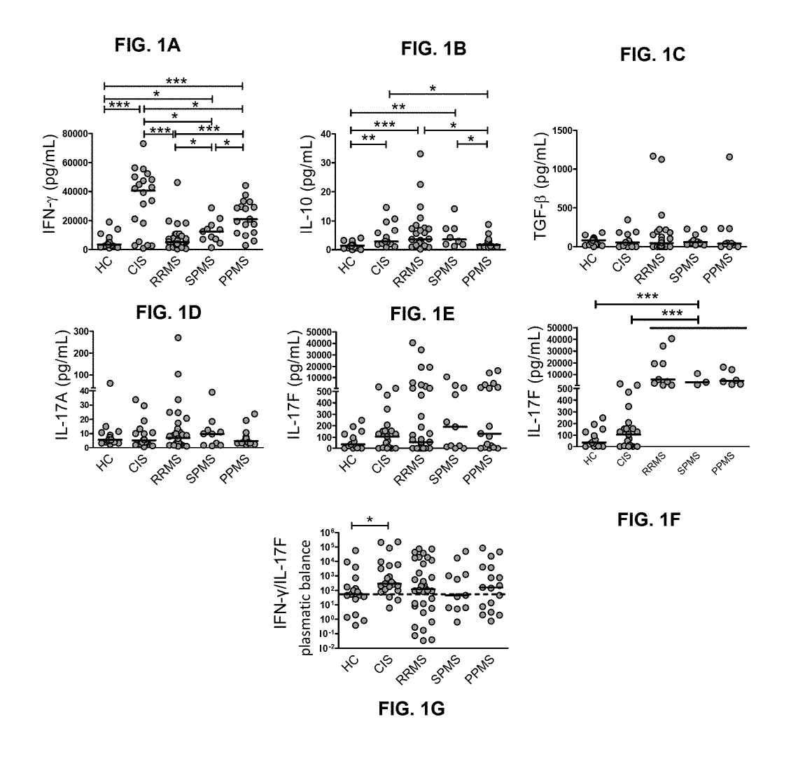

The results showed that the IFN-γ level clearly and sequencially distinguished significantly among each subtype of multiple sclerosis (FIG. 1A). CIS patients exhibited significantly higher IFN-γ production than PPMS (p=0.0349), SPMS (p=0.0219), RRMS (p=0.0004) patients and HC (p=0.0007), whereas PPMS patients had significantly increased IFN-γ level than SPMS (p=0.0305), RRMS (p<0.0001) patients and healthy controls (HC) (p=0.0001). In turn, SPMS patients showed significantly higher levels of IFN-γ than RRMS (p=0.0201) and HC (p=0.0127). Similar IFN-γ production was found in RRMS patients and healthy controls.

On the other hand, IL-10 production was similar among CIS, RRMS and SPMS patients and they were significantly higher than PPMS (p=0.0186, p=0.0149, p=0.0247, respectively) and HC (p=0.0014, p=0.0006, p=0.0080, respectively) (FIG. 1B). TGF-β levels were similar among different subtypes of patients with multiple sclerosis (FIG. 10).

When analyzing cytokines from IL-17 family, low levels of IL-17A were found in all groups of patients without significant difference between them or among patients with multiple sclerosis and healthy controls (FIG. 1D). Consistent with previous studies (Axtell R. C. and cols. Nature medicine 16.4 (2010): 406-412; and Mikulková Z. and cols. Journal of the neurological sciences 300.1 (2011): 135-141), IL-17A expression was often found below the detection limit, despite use of two different commercial kits. On the contrary, detectable concentrations of IL-17F were found in different groups of patients but there were no significant differences between them (FIG. 1E). Nevertheless, a subgroup of RRMS, SPMS and PPMS patients that expressed a plasmatic level of IL-17F greater than 500 pg/mL was significantly different to CIS patients (p<0.0001) and healthy controls (p<0.0001) (FIG. 1F).

Given the simultaneous participation of different populations of T helper lymphocytes in the development of the disease, it was determined if the ratio between cytokines produced by Th1, Th2, Th3 and Th17 lymphocytes could be a biomarker more informative and helpful than individual values of each cytokine in discriminating between patients with different subtypes of multiple sclerosis. To that end, the plasma Th1/Th17 ratio was assessed based on the ratio between plasma levels of IFN-γ and IL-17F (IFN-γ/IL-17F). Comparison between median values of Th1/Th17 ratio showed that regarding healthy controls HC (median=54.6), most of CIS patients (86% Th1; median=290.8), RRMS (65% Th1; median=120.7) y PPMS (59% Th1; median=160.8) presented a ratio Th1/Th17 more skewed toward a Th1 disease than Th17 (FIG. 1G). However, differences were significant only between CIS patients and healthy controls. In contrast, the median value of Th1/Th17 ratio in SPMS patients (54% Th17; median=45.5), was slightly skewed toward Th17 compared to healthy control patients. Medians comparison of Th1/Th17 ratio among subtypes of multiple sclerosis showed that RRMS patients had a Th1/Th17 ratio that was 2.4 times lower than CIS patients, whereas this balance was 6.4 times and 2.7 times lower in SPMS patients than in CIS and RRMS patients, respectively (FIG. 1G).

### Example 2: The Plasma IFN-γ/IL-17F Ratio in Patients with Relapsing-Remitting Multiple Sclerosis Before Initiating IFN-β Treatment Associates with Disease Activity and Progression

A clinical longitudinal study was performed in a group of RRMS patients that initiated immunomodulatory therapy with IFN-β. Before beginning with treatment, a blood sample was taken from each patient and plasma levels of Th1, Th2, Th3 and Th17 associated cytokines were measured. Moreover, neurological examination and magnetic resonance imaging (MRI) were performed to determine brain and spinal cord lesions. Then, a treatment with IFN-β was initiated and after a year of therapy the same neurological exams and MRI were repeated in all patients. With this data, Spearman statistical analyses were performed which allow determining the association or interdependence between two random variables in order to determine an association between plasma levels of cytokines and their ratios before treatment with clinical parameters. Spearman test was performed using GraphPad Prism 5.0 software and the values p<0.05 were considered statistically significant. Demographic and clinical characteristics from RRMS patients that responded positively and negatively to the treatment are shown in Table 2.

TABLE 2
Clinical and demographic characteristics of patients with
relapsing-remitting multiple sclerosis (RRMS) that respond
positively and negatively to IFN-β treatment.

| | Patients with | Patients with | |
|---|---|---|---|
| | positive response^{a} | negative response^{b} | |
| Characteristics | (n = 15) | (n = 10) | |
| Male/Female | 5/10 | 4/6 | |
| Age, mean (SD) in years | 33.2 (11.1) | 31.9 | (7.0) |
| Disease duration, | 5.0 (3.2) | 4.3 | (3.0) |
| mean in years (SD) | | | |
| Value EDSS, mean (SD) | 0.53 (0.7) | 2 | (1.6) |
| Annualized relapse rate, | 0 | 2.9 | (1.7) |
| mean (SD) | | | |
| T1-enhancing MRI lesions on | 0 | 1.9 | (1.8) |
| treatment, mean (SD) | | | |
| T2-weight MRI lesions on | 0.14 (0.36) | 3 | (1.3) |
| treatment, mean (SD) | | | |

Abbreviations: SD (Standard Deviation), EDSS (Expanded Disability Status Scale).
^{a}Patients that received treatment with IFN-β1a.
^{b}Patients that received IFN-β1a (n = 7) or IFN-β1b (n = 3).

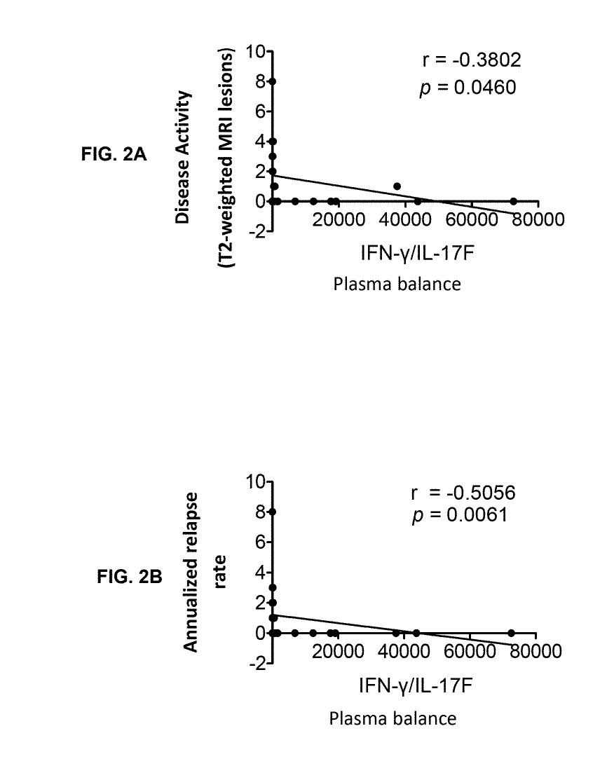

The results revealed that pretreatment plasma IFN-γ/IL-17F (Th1/Th17) ratio associated inversely with disease activity, defined as T2-weighted MRI lesions (r=−0.3802, p=0.0460), in patients with relapsing-remitting multiple sclerosis RRMS (FIG. 2A). Likewise, it was found that pretreatment plasma IFN-γ/IL-17F (Th1/Th17) ratio associated inversely with annualized rate of relapses (r=−0.5056, p=0.0061) (FIG. 2B). Taking together, these results suggest that a high plasma IFN-γ/IL-17F ratio before treatment are associated with a decrease in disease activity and relapse rate during the first year of therapy with IFN-β.

### Example 3: The IFN-γ/IL-17F Ratio in Untreated Relapsing-Remitting Multiple Sclerosis (RRMS) Patients Discriminates Between Patients that Prospectively Respond Positively or Negatively to IFN-β Treatment

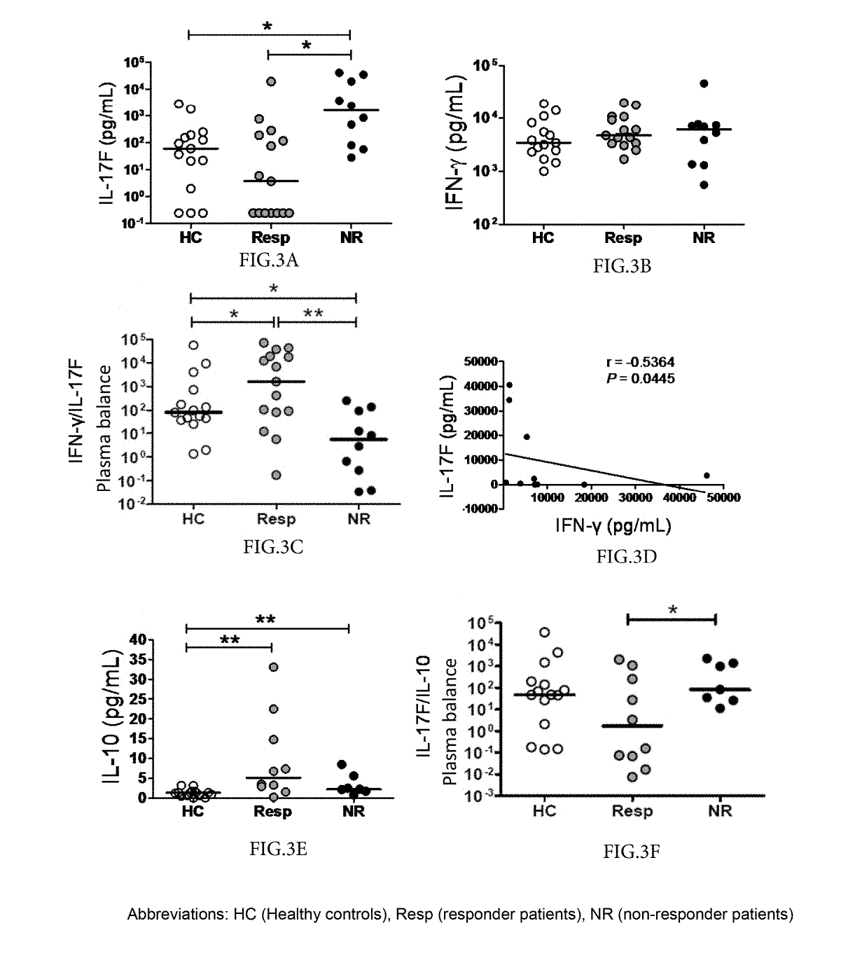

In first place, it was determined if there were differences in pretreatment plasma levels of IFN-γ, IL-17F and IL-10 and their ratios in RRMS patients that after a year of treatment with IFN-β were identified as responders (positive response) or non-responders (negative response) to the treatment (FIG. 3). The results showed that patients that responded negatively to the treatment (NR) presented IL-17F levels significantly higher than patients who responded positively to the treatment (Resp) (p=0.0141) and healthy control (HO) (p=0.0117) (FIG. 3A). No differences were detected in IFN-γ levels among different groups of patients (FIG. 3B). Remarkably, it was detected that pretreatment plasma IFN-γ/IL-17F (Th1/Th17) ratio differed in patients responding positively and negatively to the treatment with IFN-β. Patients who responded negatively to the treatment showed a pretreatment IFN-γ/IL-17F ratio significantly skewed toward Th17 in comparison with patients that responded positively to the treatment (p=0.0213) and HC (p=0.0282) (FIG. 3C). Furthermore, Spearman analysis revealed an inverse correlation between the plasma levels of IFN-γ and IL-17F in patients that responded positively to the treatment (r=−0.5364; p=0.0445), suggesting that both cytokines could have an opposite biological function in these patients (FIG. 3D). Patients that responded positively to the treatment presented pretreatment plasma levels of IL-10 higher than patients that responded negatively to the treatment and HC, but this difference only reached statistical significance regarding HC (p=0.0021) (FIG. 3E). Likewise, patients that responded negatively to the treatment presented pretreatment plasma levels of IL-10 significantly higher than HC (p=0.0090) (FIG. 3E).

The trend of patients that responded positively to the treatment of presenting higher levels of IL-10 than patients that responded negatively to the treatment it was also reflected when analyzing the IL-17F/IL-10 (Th17/Th2) ratio, which was found skewed toward Th2 and significantly lower than patients that responded negatively to the treatment (p=0.0397) (FIG. 3F).

In conclusion, these results show that the plasma levels of IL-17F, the IFN-γ/IL-17F ratio, and IL-17F/IL-10 ratio before treatment allow to discriminate between patients with relapsing-remitting multiple sclerosis (RRMS) that prospectively will result to be patients that respond positive or negatively to the treatment with IFN-8.

### Example 4: The IFN-γ/IL-17F Ratio and IL-17F/IL-10 Ratio in Untreated Patients with Relapsing-Remitting Multiple Sclerosis RRMS have a Prognostic Value for Predicting Responsiveness to Immunomodulatory Treatment with IFN-β

Based on the results described above, it was determined the prognostic value of IFN-γ/IL-17F and IL-17F/IL-10 as biomarkers predicting the IFN-β responsiveness, which was carried out through the analysis of ROC (Receiver Operating Characteristic) curves.

ROC curve is a graphic representation that illustrates the performance of a binary classification system as its threshold value of discrimination changes. The curve is created when charting true positives rate versus false positives rate at different threshold values. True positives rate is known as sensitivity, whereas the false positives rate is known as 1−specificity (Cerda J. and Cifuentes L. Revista chilena de infectología 29.2 (2012): 138-141). According to this analysis, the value of the area under the curve (AUC) is a determination of the precision of a prognosis or diagnosis test.

The accuracy of the prognostic test improves as the value of the area under the curve (AUC) distances from 0.5 and approximates to 1.0, whose significance is reflected by the value of P that results from the analysis. The fundamental characteristics of the test precision are its sensitivity (or positive predictive value, PPV) and specificity (or negative predictive value, NPV). Sensitivity (or positive predictive value, PPV) corresponds to the probability that the test correctly classifies an ill individual as positive (also known as true positives rate) whereas the specificity (or negative predictive value, NPV) corresponds to the probability that the test correctly classifies an individual not ill as negative (also known as true negatives rate). The theoretical maximum of sensitivity and specificity is 100%. Once the sensitivity and specificity are determined, it is possible to use Youden's index, defined as J=sensitivity+specificity−1 as a criterion for selecting the optimal cut-off of the test with the maximum sensitivity (PPV) and specificity (NPV) (Zou, K. and cols. Circulation 115.5 (2007): 654-657).

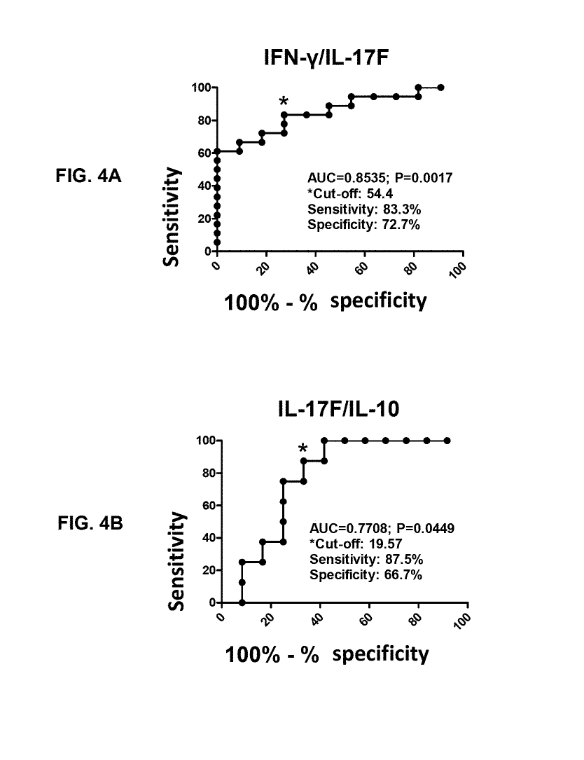

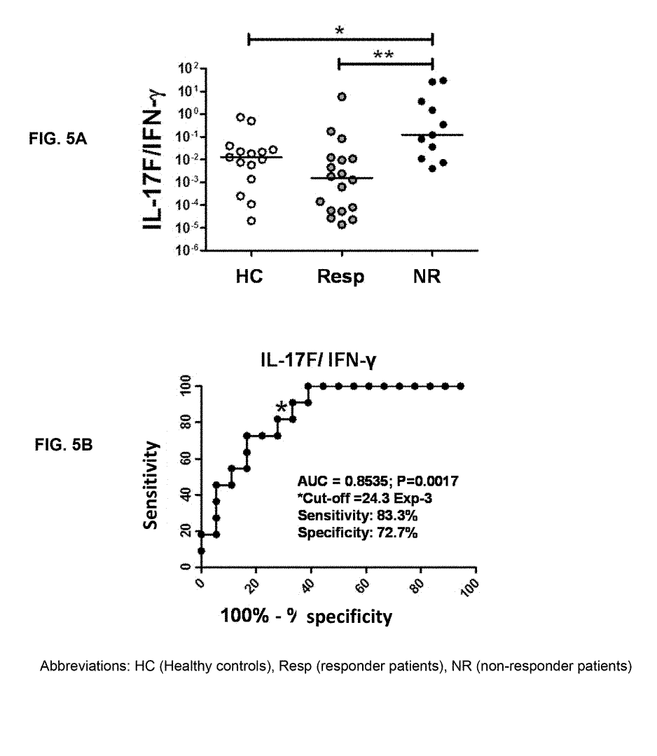

The ROC curve analysis for IFN-γ/IL-17F ratio between RRMS patients that respond positive and negatively to the treatment showed an area under the curve (AUC) of 0.8535 (p=0.0017) (FIG. 4A). According to this, it can be predicted that patients with a pretreatment plasma IFN-γ/IL-17F ratio higher or equal than 54.4 (*: cut-off value) will respond positively to IFN-β, whereas patients with IFN-γ/IL-17F ratio lower than 54.4 will respond negatively to the treatment, with a sensitivity of 83.3% and a specificity of 72.7%. In parallel, it was carried out a ROC curve analysis for the inverse ratio, that is to say, IL-17F/IFN-γ for the same patients and it was found that said ratio also has a predictive value, allowing to distinguish between patients that respond positive and negatively to the treatment (FIGS. 5A and 5B). In this way, it can be predicted that patients with a pretreatment plasma IL-17F/IFN-γ ratio higher or equal than 24.3×10⁻³ (*: cut-off value) will respond negatively to IFN-β, whereas patients with an IL-17F/IFN-γ ratio lower than 24.3×10⁻³ will respond positively to the treatment with a sensitivity of 83.3% and a specificity of 72.7%.

In the same way, the ROC curve analysis of the ratio between IL-17F/IL-10 showed an area under the curve AUC=0.7708 (p=0.0499) (FIG. 4B). According to this, it can be predicted that patients with a pretreatment plasma IL-17F/IL-10 ratio higher or equal than 19.57 will respond negatively to the treatment with IFN-β, whereas patients with IL-17F/IL-10 ratio lower than 19.57 will respond positively to the treatment with a sensitivity of 87.5% and a specificity of 66.7%.

1. An ex vivo method for predicting the response to an immunomodulatory treatment in patients with inflammatory disease CHARACTERIZED because said method comprises the following stages:
(i) to obtain a biological sample from a patient;
(ii) to quantify in said biological sample from the patient at least one predictive biomarker selected from the group that consists in the ratio between the concentrations of cytokines IFN-γ/IL-so IL-17F/IL-10, IL-17F/IFN-γ and IL-10/IL-17F;
(iii) to compare the values of the ratios between the concentrations of said cytokines of the biological sample from the patient with representative values of these ratios measured in samples of a patient's population that respond negatively to the treatment; and
(iv) to predict a positive response to the treatment based on a greater value of the ratio IFN-γ/IL-17F or IL-10/IL-17F or a lower value of the ratio IL-17F/IL-10 or IL-17F/IFN-γ in the sample from the patient, in comparison to the representative value in the samples from patient's population that respond negatively to the treatment.

2. The method according to claim 1, CHARACTERIZED because the inflammatory disease is an autoimmune disease.

3. The method according to claim 2, CHARACTERIZED because the autoimmune disease is multiple sclerosis.

4. The method according to claim 1, CHARACTERIZED because for the stage of predicting the response to the immunomodulatory treatment the following additional stages are carried out:
(i) to calculate the ratios between the concentrations of cytokines IFN-γ/IL-17F and IL-17F/IL-10 by mathematical division;
(ii) to compare ratios values with a determined cut-off value by a predictive modeling method in patient's populations that respond positive and negatively to the immunomodulatory treatment; and
(iii) to predict the positive response to the immunomodulatory treatment when the value of the ratio IFN-γ/IL-17F measured in the patient is greater than the determined cut-off value or when the value of the ratio IL-17F/IL-10 measured in the patient is lower than the determined cut-off value.

5. The method according to claim 1, CHARACTERIZED because the biological sample from the patient is selected from a group that consists in blood, blood plasma, blood serum, peripheral blood mononuclear cells, cerebrospinal fluid and urine.

6. The method according to claim 1, CHARACTERIZED because the immunomodulatory treatment includes a drug selected from the group that consists in a type I interferon, glatiramer acetate, natalizumab, fingolimod or a combination of these.

7. The method according to claim 6, CHARACTERIZED because the immunomodulatory treatment with a type I interferon is with IFN-β.

8. A kit for predicting the response to an immunomodulating treatment in patients with inflammatory disease, CHARACTERIZED because comprises:
(i) means to obtain a biological sample from the patient;
(ii) means to quantify in a biological sample from the patient at least one predictive biomarker selected from the group that consists in the ratios between the concentrations of cytokines IFN-γ/IL-17F, IL-17F/IL-10, IL-17F/IFN-γ and IL-10/IL-17F;
(iii) means to compare the value of the ratios between the concentrations of said cytokines of the biological sample from the patient with representative values of these ratios measured in samples of a patient's population that respond negatively to the treatment;
(iv) means to predict a positive response to the treatment based on a greater value of the ratio IFN-γ/IL-17F or IL-10/IL-17F or a lower value of the ratio IL-17F/IL-10 or IL-17F/IFN-γ in the sample of the patient, in comparison to the representative value in the sample of the patient's population that respond negatively to the treatment; and
(v) a brochure with instructions to use said means.

9. The kit according to claim 8, CHARACTERIZED because the inflammatory disease is an autoimmune disease.

10. The kit according to claim 9, CHARACTERIZED because the autoimmune disease is multiple sclerosis.

11. The kit according to claim 8, CHARACTERIZED because the biological sample from the patient is selected from a group that consists in blood, blood plasma, blood serum, peripheral blood mononuclear cells, cerebrospinal fluid and urine.

12. The kit according to claim 8, CHARACTERIZED because the immunomodulatory treatment includes a drug selected from the group that consists in a type I interferon, glatiramer acetate, natalizumab, fingolimod or a combination of these.

13. The kit according to claim 12, CHARACTERIZED because the immunomodulatory treatment with a type I interferon is with IFN-β.
